(19) United States
(12) Patent Application Publication
(10) Pub. No.: US 2011/0020428 A1
(43) Pub. Date: Jan. 27, 2011

(54) GEL-STABILIZED LIPOSOME COMPOSITIONS, METHODS FOR THEIR PREPARATION AND USES THEREOF

(76) Inventor: Qun Zeng, Calgary (CA)

Correspondence Address: Bell & Manning, LLC, 122 E. Olin Avenue, Suite 290, Madison, WI, 53713, US

(21) Appl. No.: 12/742,735

(22) PCT Filed: Nov. 13, 2008
(86) PCT No.: PCT/CA2008/001994

§ 371 Date: Sep. 21, 2010

Related U.S. Application Data
(60) Provisional application No. 60/988,214, filed on Nov. 15, 2007.

(51) Int. Cl. A61K 9/127; A61K 38/21; A61K 31/7048; A61K 38/42; A61K 31/4355; A61K 31/704; A61K 31/337

(52) U.S. Cl. 424/450; 424/85.7; 514/31; 514/13.5; 514/280; 514/34; 514/449

(57) ABSTRACT

Compositions, preparation methods and potential applications of gel-stabilized liposomes with high degree of entrapment efficiency and stability are described. In particular, the novel liposome system comprises liposomes that each encapsulate an internal thermo-transformable hydrogel, dispersed and suspended in a continuous external thermo-reversible hydrogel phase. Agents, such as active agents, are encapsulated in the internal hydrogel core or in the lipid bilayer, or multilayers, depending on whether the active agent is water or lipid soluble, respectively.

### FIELD OF DISCLOSURE

The present disclosure relates to novel liposomal compositions having a high encapsulation efficiency and stability. In particular the present disclosure relates to gel-stabilized liposome compositions, methods for preparing these compositions and their use, in particular for drug delivery.

### BACKGROUND

A wide variety of therapeutic and diagnostic formulations that may be characterized as ‘particulate nanomedicines’ have been developed since the identification of liposomes in the mid 1960's (Bangham et al., 1965, J. Mol. Biol. 13:238-252). Much of the work in this field has been devoted to improving the efficiency with which selected compounds are encapsulated (the entrapment efficiency) within these particles, as well as optimizing the stability and modulating the size of the particles (see for example the following patents and publications: U.S. Pat. No. 4,089,801, U.S. Pat. No. 4,235,871, U.S. Pat. No. 4,522,803, U.S. Pat. No. 4,708,861, U.S. Pat. No. 4,740,375, U.S. Pat. No. 4,761,288, U.S. Pat. No. 6,221,387, U.S. Pat. No. 5,008,109, EP162764, U.S. Pat. No. 5,064,655, U.S. Pat. No. 5,230,899, U.S. Pat. No. 6,221,387, U.S. Pat. No. 6,048,546, U.S. Pat. No. 6,284,375, U.S. Pat. No. 6,331,315, WO89/02267, WO03/075888, US20020048598, US2003/0180348, US2006/0171990 and WO2006/065234). A few drug encapsulated liposome formulations have reached clinical use, including, for example, adriamycin (liposomesDoxil®, amphotericin (AmBisome®) and daunomycin (DaunoXome®).

To produce liposomes for commercial disclosures, the following characteristics are desirable:
1) high degree of encapsulation,
2) final product obtainable by a simple procedure,
3) preparation on a large scale,
4) long term storage stability, and
5) uniform and easily-controlled size and size distribution.

Various techniques have been devised to improve the above commercially-desirable characteristics of liposomal formulations (see Gao & Huang, Biochim. Biophy. Acta 1987, 897:377-378; Haran et al. Biochim. Biophy. Acta 1993, 1151:201-215; Mayer et al. Biochim. Biophy. Acta 1990, 1025:143-151). However, the various previously reported liposome preparations do not provide both good stability and high active agent encapsulation yield (desirably around 90% to 100%) without modifying the active agents.

### SUMMARY OF THE DISCLOSURE

Described herein is a novel gel-stabilized liposome composition which exhibits significant advancement in drug encapsulation yield (up to 100%), liposome stability as well as uniform and flexible vesicle sizes. The compositions of the present disclosure comprise liposomes having an internal phase composed of an internal thermo-transformable hydrogel. Further stabilization is imparted to the liposomal compositions of the present disclosure by dispersing the liposomes in an external phase comprising an external thermo-reversible hydrogel.

Accordingly, the present disclosure includes a gel-stabilized liposome composition comprising liposomes having an internal phase and an external phase, wherein the internal phase comprises an internal thermo-transformable hydrogel and the external phase comprises an external thermo-reversible hydrogel and the liposomes are dispersed in the external phase.

The present disclosure also includes a process for the preparation of the liposomal compositions described herein. In an embodiment of the disclosure, the process comprises:

(a) preparing or obtaining a hydrosol comprising one or more internal thermo-transformable hydrosols and, optionally, one or more water-soluble agents wherein the hydrosol is prepared in an aqueous medium;
(b) preparing or obtaining a solution comprising one or more lipids and, optionally, one or more lipid-soluble agents in an organic solvent that is substantially immiscible with the aqueous medium;
(c) combining the solution of (a) with the solution of (b) at a temperature which is higher than the sol-gel phase transition temperature of the one or more internal thermo-transformable hydrosols and under conditions to produce an emulsion;
(d) lowering the temperature of said emulsion of (c) to below the sol-gel phase transition temperature of the one or more internal thermo-transformable hydrosols to transform said one or more hydrosols into one or more hydrogels in said emulsion;
(e) optionally removing a portion of the organic solvent from the emulsion of (d) at a temperature lower than the sol-gel phase transition temperature of the one or more internal thermo-transformable hydrogels; and
(f) combining the emulsion of (d) or (e) with one or more external thermo-reversible hydrogels and removing any remaining organic solvent, wherein said combining and said removal of solvent is at a temperature lower than the sol-gel phase transition temperature of the one or more internal thermo-transformable hydrogels and under conditions to form a homogeneous dispersion of liposomes in the one or more external thermo-reversible hydrogels, wherein said one or more external thermo-reversible hydrogels are prepared in an aqueous medium and the liposomes have an internal phase comprised of the one or more internal thermo-transformable hydrogels.

The present disclosure further includes methods of using the liposome compositions of the present disclosure for example, for delivery of agents to a cell, tissue and/or subject. Accordingly the present disclosure includes a method for delivering a one or more agents to a biological system comprising administering a gel-stabilized liposome composition of the present disclosure to said system, wherein the gel-stabilized liposome composition comprises the agent.

Also included in the present disclosure is a method of delivering an active agent to a subject in need of treatment with the active agent comprising administering an effective amount of a gel-stabilized liposome composition of the present disclosure to said subject, wherein the gel-stabilized liposome composition comprises the active agent.

Also included in the present disclosure is a use of a gel-stabilized liposome composition of the present disclosure for delivery of agents to a cell, tissue or subject as well as a use of a gel-stabilized liposome composition of the present disclosure to prepare a medicament for delivery of agents to a cell, tissue or subject. Also included is a gel-stabilized liposome composition for use to deliver agents to a cell, tissue or subject. In each of these uses, the gel-stabilized liposome composition comprises the agent, suitably an active agent.

The present disclosure further includes a pharmaceutical composition comprising a gel-stabilized liposome composition of the present disclosure and a pharmaceutically acceptable carrier. In an embodiment, the gel-stabilized liposome composition comprises an agent, suitably an active agent.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The present disclosure will be better understood with reference to the enclosed drawings illustrating particular embodiments of said disclosure. More particularly, said drawings comprise the following figures:

FIG. 1 shows a Transmission Electron Micrograph (TEM) of a gel-stabilized liposome composition containing amphotericin B in accordance with one embodiment of the present disclosure.

FIG. 2 is a graph showing the plasma amphotericin B concentration-versus-time for five rats receiving a single 1 mg/kg intravenous dose of gel-stabilized liposome composition loaded with amphotericin B in accordance with one embodiment of the present disclosure, compared with the control, DAMB.

FIG. 3 is a bar graph showing the distribution of amphotericin B in various tested tissues after administration of gel-stabilized liposome composition loaded with amphotericin B in accordance with one embodiment of the present disclosure.

FIGS. 4A and 4B show a particle size distribution analysis of gel-stabilized liposome composition loaded with bovine hemoglobin prepared using ether (FIG. 4A) or methyl tert-butyl ether (FIG. 4B) as the organic solvent in accordance with one embodiment of the present disclosure.

### DETAILED DESCRIPTION

### I. Definitions

A liposome is a spherical vesicle having a surface membrane composed one or more lipid bilayers. The liposome membrane is composed of a single lipid bilayer or several lipid bilayers (multilayered). In an embodiment, the lipid bilayer is composed of phospholipids and cholesterol. Liposomes can be composed of naturally-derived phospholipids with mixed lipid chains or of pure surfactant components. The additional lipid layers of the multilayered membranes further enhance the stability of the liposome vesicles by strengthening the structural integrity of the vesicles.

In the context of the present disclosure, a “gel phase” has its usual meaning, a semisolid elastic material in which the movement of the material is restricted. The term “sol” as used herein refers to the solution or liquid phase of a material. When solvating media are aqueous, the sols and gels formed therein are be referred to as hydrosols and hydrogels, respectively.

The term “agent” as used herein refers to any substance which one wishes to encapsulate in the liposomes of the present disclosure. Typically the agent will be a biologically active agent or a drug, and includes, for example, small organic molecules, small inorganic molecules, oligonucleotides, sugars, carbohydrates, proteins, peptides and lipids.

The term “substantially” as used herein means that the referred-to condition is met with the possible existence of minor, for example, less than 5%, suitably less than 1%, of alternative conditions. For example, the term “substantially immiscible” means that two substances do not dissolve in or mix with each other to the extent that less than 5%, suitably less than 1%, of the substances are dissolved in or mix with each other.

The term “pharmaceutically acceptable” means suitable for or compatible with the treatment of subjects, including humans.

The term “biomolecule compatible” or “bio-compatible” as used herein means that a substance either stabilizes proteins and/or other biomolecules against denaturation or does not facilitate their denaturation.

The term “subject” as used herein includes all members of the animal kingdom, including mammals, in particular, humans.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### II. Liposome Compositions

The disclosure in the present disclosure relates to a bio-compatible gel-stabilized liposome composition with a high degree of encapsulation efficiency and stability, its preparation method and uses.

Accordingly, the present disclosure includes a gel-stabilized liposome composition comprising liposomes having an internal phase and an external phase, wherein the internal phase comprises an internal thermo-transformable hydrogel and the external phase comprises an external thermo-reversible hydrogel and the liposomes are dispersed in the external phase.

According to present disclosure, both the internal and external hydrogels of the disclosure have thermo-reversible properties in that they become a gel upon cooling and become a sol upon heating above a certain temperature. This property is useful in that it meets the requirements of preparation processes and clinical uses of the liposomes of the disclosure by ensuring the stability needed for long-term storage while making the active agent readily available for immediate administration. At human body temperature, the internal thermo-transformable hydrogel will be in either a gel or sol state while the external thermo-reversible hydrogel phase will only be in a sol state. However, both the internal thermo-transformable hydrogel and external thermo-reversible hydrogels should be in a gel state at a storage conditions.

The hydrogel in the internal phase of the liposome and the hydrogel forming the external thermo-reversible hydrogel are the same or different, depending on the desired properties of the liposomes. In alternative embodiments, suitable hydrogels for the internal thermo-transformable hydrogel and the external thermo-reversible hydrogel are, for example, natural, semi-synthetic or synthetic, and are suitably biodegradable and biocompatible.

The internal thermo-transformable hydrogel is thermo-reversible or thermal irreversible. It need only be able to transform from the sol to the gel state upon lowering the temperature below its sol-gel phase transition temperature. The sol-gel transition temperature, depends on the concentration or modifications of the hydrogels, or properties of the solvating media. Chemical modification, for example, includes, for example, the addition of modifying groups to the hydrogels or the introduction of cross-linking agents to the solvating media. Other examples of chemical modifications include modulating the chemical makeup, pH, osmotic pressure or ionic strength of the internal and external solutions.

In a particular embodiment, the internal hydrogel or external hydrogel are gelatin, and the aqueous media are water. A variety of gelatins can be selected for use in the compositions of the present disclosure, these gelatins generally comprising a heterogeneous mixture of single or multi-stranded polypeptides, each with extended left-handed proline helix conformations, and containing on average between 300 to 4000 amino acids. Gelatins typically contain a large number of glycine, proline and 4-hydroxyproline residues. A gelatin hydrosol generally comprises solvated gelatin molecules interpenetrated by water. Gelatin hydrosols can be adapted to form elastic thermo-reversible hydrogels. The sol-gel transition temperature of gelatin solution will vary, for example, depending on the concentration of the gelatin, modifications of the gelatin, and the composition of the solvating medium.

In embodiments using gelatin as the internal hydrogel and the external hydrogel phases and using water as the aqueous medium, the chemical properties of gelatin, and the resultant hydrosols, are tailored for a particular application as would be known to a person skilled in the art. For example, gelatin having a higher triple-helix content generally swells to a lesser extent in water, and the resulting hydrogel formed from the hydrosol therefore generally is stronger compared to the gel formed from a gelatin having a lower triple-helix content. Gelatins for use in the disclosure are optionally modified, for example by the addition of cross-linking agents, such as transglutaminase to link lysine residues to glutamine residues, or glutaraldehyde to link lysine residues to lysine residues.

In an embodiment of the disclosure, the internal and/or external hydrogels are selected from gelatin and agarose. In another embodiment the internal hydrogel is agarose and the external hydrogel is gelatin. In a further embodiment, the internal hydrogel and external hydrogels are both gelatin.

In an embodiment of the present disclosure, various agents are encapsulated into the liposomes. In an embodiment of the disclosure the agent is an active agent. Active agents include, for example, natural, semi-synthetic or synthetic drugs. For therapeutic and diagnostic use, the active agents include, for example, a drug, a polynucleotide, a polypeptide, a protein, an antigen, a nutrient and a flavor substance, but are not limited to these. Agents with different soluble properties can be encapsulated in different locations within the liposomes of the present disclosure. Water-soluble agents are encapsulated within the internal hydrogel phase while lipid-soluble agents are encapsulated within the lipid bilayer.

In some embodiments, in order to encapsulate agents in different locations within the liposomes of the present disclosure, water-soluble agents are dissolved and dispersed in the internal thermo-transformable hydrogel before it is converted to its gel form and lipid-soluble agents are dissolved in the lipid organic solution. In some embodiments, agents are covalently or noncovalently linked to the internal hydrogel or to the lipids. The ratio of the agent to the internal hydrogel core is controlled, for example, so that it does not significantly hinder the sol-gel transition process.

In an embodiment of the present disclosure, liposome-forming molecules include lipids. One or more naturally occurring and/or synthetic lipid compounds are used in the preparation of the liposomes. In the present disclosure, suitable lipids are, for example, phospholipids, such as natural, or synthetic phospholipids, saturated or unsaturated phospholipids, or phospholipid-like molecules, but are not limited to these. Representative suitable phospholipids or lipid compounds include, but are not limited to, soybean lecithin, egg lecithin, lethicin, lysolecithin, phosphatidylserine, phosphatidylethanolamine, phosphatidylcholine and phosphatidylinositol, and the like. Additional non-phosphorous-containing lipids include, but not limited to, stearylamines, fatty acids, fatty acid amides and the like. In an embodiment of the present disclosure, the phospholipids are mixed with a sterol such as cholesterol to stabilize the phospholipid bilayer or multilayer. In other embodiments, the lipid is chemically or physically modified. Modifications function, for example, to alter the properties of the lipid and of the resulting liposome vesicles. Methods of modifying lipids are known in the art of liposomal formulations.

### III. Processes for Preparation

The present disclosure also includes processes for the preparation of the liposomal compositions described herein. The process comprises:
(a) preparing or obtaining a hydrosol comprising one or more internal thermo-transformable hydrosols and, optionally, at least one water-soluble agent wherein the hydrosol is prepared in an aqueous medium;
(b) preparing or obtaining a solution comprising one or more lipids and, optionally, one or more lipid-soluble agents in an organic solvent that is substantially immiscible with the aqueous medium;
(c) combining the solution of (a) with the solution of (b) at a temperature which is higher than the sol-gel phase transition temperature of the one or more internal thermo-transformable hydrosols and under conditions to produce an emulsion;
(d) lowering the temperature of said emulsion of (c) to below the sol-gel phase transition temperature of the one or more internal thermo-transformable hydrosols to transform said one or more hydrosols into one or more hydrogels in said emulsion;
(e) optionally removing a portion of the organic solvent from the emulsion of (d) at a temperature lower than the sol-gel phase transition temperature of the one or more internal thermo-transformable hydrogels; and
(f) combining the emulsion of (d) or (e) with one or more external thermo-reversible hydrogels and removing any remaining organic solvent, wherein said combining and said removal of solvent is at a temperature lower than the sol-gel phase transition temperature of the one or more internal thermo-transformable hydrogels and under conditions to form a homogeneous dispersion of liposomes in the one or more external thermo-reversible hydrogels, wherein said one or more external thermo-reversible hydrogels are prepared in an aqueous medium and the liposomes have an internal phase comprised of the one or more internal thermo-transformable hydrogels.

In the process of the present disclosure, the organic solvents suitable for dissolving the lipids in (b) of the process include any solvent in which the lipids are substantially soluble and which is substantially immiscible with the aqueous media used for forming the internal hydrogels and, include, but are not limited to, ethers, such as diethyl ether, di-n-butyl ether and methyl tertiary butyl ether (MTBE), cyclohexane and chloroform and combinations thereof. The lipids are used at any concentration that is operable to form at least one bilayer, including multilayers, encapsulating the inner hydrogel.

In an embodiment of the process of the present disclosure, the “conditions to produce an emulsion” in (c) comprise adding the solution comprising one or more thermo-transformable hydrosols into the lipid organic solution in a suitable ratio, followed by a mixing, for example by mechanical dispersion, to form an emulsion. This emulsion is a “hydrosol-in-oil” emulsion in which the hydrosol from (a) is dispersed in the organic solvent in the form of individual droplets. In particular embodiments, the lipid organic solution is used in amounts excess to the one or more thermo-transformable hydrogels. Non-limiting examples of suitable ratios of the lipid organic solution to the thermo-transformable hydrogel are approximately 3:1 to 15:1, suitably 4:1 to 10:1, more suitably 5:1 to 8:1, or about or between any integer value or values within these ranges.

A person skilled in the art would be able to select suitable temperatures and conditions to convert any thermally-transformable hydrosol to its corresponding hydrogel based on the sol-gel phase transition temperature of the thermo-transformable hydrogel. In embodiments, when using gelatin as the internal thermo-transformable hydrogel, the hydrosol form is converted to the hydrogel form by cooling the emulsion of (c) to a suitable temperature which is below the sol-gel phase transition temperature of gelatin, wherein the suitable temperatures for cooling is in the range of approximately 0° C. to 18° C., suitably 2° C. to 12° C., more suitably 4° C. to 8° C., or about or between any integer value or values within these ranges.

In embodiments using agarose as the internal thermo-transformable hydrogel, the hydrosol form is converted to the hydrogel form by cooling the emulsion of (c) to a suitable temperature which is below sol-gel phase transition temperature of agarose, wherein the suitable temperature for cooling is in the range of approximately 0° C. to 30° C., suitably 2° C. to 20° C., more suitably 4° C. to 15° C., or about or between any integer value or values within these ranges.

According to embodiments of the present disclosure, the organic solvent is at least partially removed after formation of emulsion of (d). The removal of the organic solvent is desirably done at a temperature below the sol-gel phase transition temperature of the one or more internal thermo-transformable hydrogels and is typically performed under reduced atmosphere. Sufficient organic solvent is removed, for example, to obtain a suitable volume ratio of the emulsion of (d) to aqueous medium comprising the external thermo-reversible hydrogel (i.e. the external hydrogel solution). Suitable volume ratios of the emulsion to the external hydrogel solution are, for example, in the range of about 3:7 to about 8:2, suitably about 2:3 to about 3:2, more suitably about 1:1.

In still further embodiments of the disclosure an amount of the aqueous medium, optionally comprising the external thermo-reversible hydrogel is added into the emulsion of (d) either before or following evaporation of a portion of the organic solvent. In an embodiment of the disclosure, the addition of the external hydrogel solution is performed following evaporation of a portion of the organic solvent from the emulsion of (d).

Suitably the addition of the external thermo-reversible hydrogel solution is done at a temperature below the sol-gel phase transition temperature of the one or more internal thermo-transformable sol gels followed by mixing, for example by stirring. Suitably the concentration of the external hydrogel solution added in this embodiment of the process of the disclosure is in the range of about 0% to about 1% (w/v), more suitably about 0.1% to about 0.5% (w/v), even more suitably about 0.4% to about 0.49% (w/v). In this embodiment, any remaining organic solvent is removed following addition of the external hydrogel solution. The remaining solvent is again suitably removed at a temperature below the sol-gel phase transition temperature of the one or more internal thermo-transformable sol gels and under reduced pressure. Following removal of the remaining organic solvent, a final external hydrogel solution is added, suitably at a concentration in the range of about 20% to about 40% (w/v), more suitably about 30% (w/v), and at a temperature below the sol-gel phase transition temperature of the one or more internal thermo-transformable sol gels, to provide a final external hydrogel concentration in the liposomal composition of about 2% to about 5% (w/v), suitably about 3% (w/v), or a concentration that ensures that the external phase of the liposomal composition of the present disclosure forms a hydrogel state at the desired temperature of storage. This series of steps involving addition of the external hydrogel solution, removal of organic solvent and addition of a final amount of external hydrogel solution are suitably performed under conditions, for example with mixing, at concentrations and temperatures, to form a homogeneous dispersion of liposomes in the one or more external thermo-reversible hydrogels, wherein the liposomes have an internal phase comprised of the one or more internal thermo-transformable hydrogels.

In another embodiment of the process of the present disclosure, the external hydrogel solution at a concentration of about 0.01% to about 1% (w/v) is added prior to removal of any of the organic solvent followed by removal of all of the organic solvent under conditions, for example with mixing, at concentrations and temperatures, to form a homogeneous dispersion of liposomes in the one or more external thermo-reversible hydrogels, wherein the liposomes have an internal phase comprised of the one or more internal thermo-transformable hydrogels. In this embodiment, a sufficient volume of the external hydrogel solution is used to ensure the proper formation of liposomes, said volume being at least equal to or exceeding that of the organic solvent present in the emulsion of (c).

In process of the present disclosure, gelation stabilized liposomes with a diameter ranging from about 30 nm to about 3000 nm are prepared, the liposomes having a single lipid bilayer or multiple lipid bilayers (multilayered). The size of liposomes is controlled by, for example, the volume and concentration of the solutions used and the intensity of the energy used during the mixing of the solutions. In general, the greater the energy and duration of the mixing, the smaller and more uniform the size of the inner hydrogel droplets and, hence, the smaller and more uniform size of the resulting liposomes of the present disclosure. Further, the larger the volume of solutions used, in particular the larger the volume of the external hydrogel solution used, the smaller the size of the liposomes formed. A person skilled in the art would be able to vary the above parameters to obtain the size of the liposomes that are desired to be formed.

In particular embodiments, mixing and combining of solutions and emulsions is done by mechanical dispersion methods that include, but are not limited to, ultrasonicating, homogenizing, vigorous mixing, agitating, vortexing, or a combination thereof. The size of the droplets of the internal hydrosol, and accordingly the size of the liposomes, are, for example, controlled by modulating the strength and duration of ultrasonication or homogenization etc., as discussed above.

The choice of gelation-stablized liposomes with a desirable average size and size distribution is dictated by the use for the compositions of the disclosure. For example, if the composition of the disclosure comprising the agent were to circulate in the blood stream for an extended time, liposomes having a smaller diameter, such as 100 nm, and narrower size distribution would be desirable. If the composition of the disclosure comprising agent were to concentrate in spleen or liver, a larger size would be more desirable.

In other embodiments of the present disclosure, osmotic regulating agents, for example, but not limited to, sodium chloride, glycerin, mannitol and/or glucose, pH regulation agents and/or other additives, are added to the said internal hydrosol solution in (a) or external hydrosol solution in (f) but are not essential to the formation and stability of the liposomes of the present disclosure.

### IV. Uses

The liposomal compositions of the present disclosure are new therefore the present disclosure includes all uses of said compositions, including uses related to medical therapies, diagnostics, and analytical tools. In particular the liposomal compositions are useful, for example, as a drug carrier, a blood cell substitute, a vaccine carrier, in protein separation and for enzyme immobilization. In these contexts, the liposomal compositions of the present disclosure are expected to be superior to conventional liposomes as they possess enhanced mechanical stability, controllable size, increased loading capacity and simplified preparation on a large scale.

The present disclosure therefore includes methods of using the liposome compositions of the present disclosure, for example, for delivery of agents to a cell, tissue and/or subject. Accordingly the present disclosure includes a method for delivering a one or more agents to a biological system comprising administering a gel-stabilized liposome composition of the present disclosure to said system, wherein the liposome compositions comprises the agent.

Also included in the present disclosure is a method of delivering an active agent to a subject in need of treatment with the active agent comprising administering an effective amount of a gel-stabilized liposome composition of the present disclosure to said subject, wherein the liposome compositions comprises the active agent.

Also included in the present disclosure is a use of a gel-stabilized liposome composition of the present disclosure for delivery of agents to a cell, tissue or subject as well as a use of a gel-stabilized liposome composition of the present disclosure to prepare a medicament for delivery of agents to a cell, tissue or subject. Also included is a gel-stabilized liposome composition for use to deliver agents to a cell, tissue or subject. In each of these uses, the gel-stabilized liposome composition comprises the agent, suitably an active agent.

The term “effective amount” of a composition of the present disclosure is a quantity sufficient to, when administered to the subject, including a mammal, for example a human, effect beneficial or desired results, including clinical results and diagnostic results, and, as such, an “effective amount” or synonym thereto depends upon the context in which it is being applied. For example, in the context of treating a disease, disorder or condition, it is an amount of the composition sufficient to achieve such a treatment as compared to the response obtained without administration of the composition. As a further example, in the context of diagnosing or detecting a disease, disorder or condition, it is an amount of the composition sufficient to achieve such a diagnosis as compared to the response obtained without administration of the composition. The amount of a given composition of the present disclosure that will correspond to such an amount will vary depending upon various factors, such as the given active agent in the composition, the pharmaceutical formulation, the route of administration, the type of disease, disorder or condition, the identity of the subject or host being treated, and the like, but can nevertheless be routinely determined by one skilled in the art.

Moreover, a “treatment”, “prevention” or diagnostic regime of a subject with an effective amount of the composition of the present disclosure consists, for example, of a single administration, or alternatively comprise a series of applications. For example, the composition of the present disclosure is administered at least once a week. However, in another embodiment, the composition is administered to the subject from about one time per week to about once daily for a given treatment. The length of the treatment period depends on a variety of factors, such as the severity of the disease or disorder, the age of the patient, the concentration and the activity of the active agents in the composition of the present disclosure, or a combination thereof. It will also be appreciated that the effective dosage of the composition used for the treatment or prophylaxis is optionally increased or decreased over the course of a particular treatment or prophylaxis regime. Changes in dosage result and become apparent by standard diagnostic assays known in the art. In some instances, chronic administration is required. It will also be appreciated that, for diagnostic applications, the compositions of the disclosure are only administered once, for example, prior to the diagnostic assay.

As used herein, and as well understood in the art, “treatment” is an approach for obtaining beneficial or desired results, including clinical results. Beneficial or desired clinical results include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” also means, for example, prolonging survival as compared to expected survival if not receiving treatment.

In further embodiments, the liposomal compositions of the present disclosure may be adapted for delivery to subjects via known routes of administration, such as, for example, intravenously, intramuscularly, intraperitoneally, orally, subcutaneously, ophthalmally, and percutaneously. The compositions of the disclosure are also formulated in a variety of dosage forms, for example as ointments, suspensions, powders, tablets and capsules. Dosages of the compositions of the disclosure are tailored to individual needs, the desired effect, and the chosen route of administration. The compositions containing the compositions of the disclosure are prepared by known methods for the preparation of pharmaceutically acceptable compositions which are administered to subjects, such that an effective quantity of the active substance is combined in a mixture with a pharmaceutically acceptable vehicle. Suitable vehicles are described, for example, in Remington's Pharmaceutical Sciences (2003—20th edition) and in The United States Pharmacopeia: The National Formulary (USP 24 NF19) published in 1999. On this basis, the compositions include, albeit not exclusively, solutions of the liposomes in association with one or more pharmaceutically acceptable vehicles or diluents, and contained in buffered solutions with a suitable pH and iso-osmotic with the physiological fluids.

The present disclosure further includes a pharmaceutical composition comprising a gel-stabilized liposome composition of the present disclosure and a pharmaceutically acceptable carrier. In an embodiment, the gel-stabilized liposome composition comprises an agent, suitably an active agent.

In embodiments of the disclosure, the compositions of the disclosure are introduced or incorporated into medical devices for delivery to a specific treatment site, or for controlled release. Alternative uses of the compositions of the disclosure include, but are not limited to: cell replacement therapies, for example, red blood cell replacement; stabilizers for protein and peptide-based drugs and therapeutics, for example by stabilizing such compounds to reduce aggregation and/or precipitation of these macromolecules; vaccine carriers, for example to improve the shelf life of peptides vaccines; immunologic adjuvants, for example to activate phagocytosis by macrophages; cell conjugation; gene therapy; gene transfection; or; in diagnostic disclosures.

In alternative embodiments, the compositions of the disclosure are stored under conditions where both the internal thermo-transformable hydrogel core and the external thermo-reversible hydrogel are in a gel state. When the compositions of the disclosure are administered, however, the external hydrogel phase is in the sol state while the inner core is either in sol or gel state.

For example, in embodiments of the present disclosure, it is suitable to store product comprising a composition of the disclosure at a temperature lower than the sol-gel transition temperature of the internal thermo-transformable hydrogel and the external thermo-reversible hydrogel, at which temperature, the internal hydrogel and the external hydrogel are both in the gel state. In other embodiments, the compositions of the disclosure are stored as a dehydrated powder prepared by drying, such as, but not limited to, lyophilization or spraying and are, optionally, subsequently hydrated in vitro or in vivo.

In alternative embodiments, two or more compositions of the disclosure are mixed together, for example in a single dosage form, to facilitate the use of the compositions of the disclosure via a particular delivery route, or in particular therapeutic or diagnostic disclosures.

### EXAMPLES

Non limitative examples of the disclosure are provided hereinafter to illustrate embodiments of the disclosure

### Example 1

### Preparation of Empty Gel-Stabilized Liposome Composition with a High Degree of Entrapment Efficiency and Stability

### (a) Materials and Methods

A gelatin solution having a concentration of about 4% (w/v) was prepared by dissolving 1.2 g of gelatin B 250 (from Sigma) in 30 ml of distilled water at about 40° C.

A lipid solution was prepared by dissolving 4 g of soybean lecithin (from Shanghai Taiwei Pharmaceutical Ltd, China) and 1.25 g of cholesterol (from Sigma) in 180 ml of diethyl ether.

The 30 ml gelatin solution was incorporated into 180 ml of the lipid solution at a temperature in the range of 25-30° C. and sonicated by probe sonicator (JY92-2D, Scientz Biotechnology Co. Ltd., Ningbo, China) for about 10 min to form a homogenous and translucent emulsion, which did not separate within 15 min following sonication, and in which ether was in continuous phase. The emulsion was subsequently placed in an ice-water bath at about 4° C. to 8° C. to transform each of the droplets of gelatin sol into a gelatin gel core.

At least a portion of the ether present in the hydrogel-in-oil emulsion was removed from the cooled emulsion by rotary evaporation under vacuum at 4° C. to 8° C., a temperature below the sol-gel transition temperature of the gelatin. Gelatin (2.1 g) in distilled water (70 mL) were added while stirring. Removal of the organic solvents was continued until the last trace of the organic solvents was gone. A translucent dispersion was obtained. This dispersion was homogenized by sonication for 3 sec (200 W) to provide an empty gel-stabilized liposome composition. This composition was stored at about 4° C. to 8° C., or as a dehydrated powder, which may be rehydrated in vitro or in vivo. The powder may for example be produced by spray-drying the gel-stabilized liposome vesicle system at an inlet temperature of about 100° C. and an outlet temperature of about 60° C., using a spray at a rate of about 1.9 to 2.1 ml/min and pressure of about 17 to 18 kPa (SD-1000, Tokyo RiKaKiKai Co. Ltd., Japan)

### (b) Characterization of Liposome Size

A laser diffraction particle analyzer (L230, Beckman Coulter, USA) was used to determine the size of the liposomes of the present disclosure formed under the above-described conditions. A sample of the empty gel-stabilized liposome was added into a sample cell containing normal saline having a refractive index of 1.333 until a polarization intensity differential scattering (PIDS) obscuration of 40% was obtained. All data were collected over a period of 120 s. The empty gel-stabilized liposome vesicle system of the disclosure was found to comprise liposomes having an average diameter of approximately 101 nm±33 nm.

Examples 2 to 6 describe the preparation of the gel-stabilized liposome compositions of the present disclosure encapsulating various active agents in the internal hydrogel core or lipid bilayer or multilayers. Table 1 summarizes the experimental protocols discussed in detail below, and the results obtained with respect to the entrapment efficiency of the gel-stabilized liposome compositions of the disclosure for various active agents and the liposome size. The encapsulation of active agents of up to about a 100% may be obtained as is shown in the examples. As the data in Table 1 indicate, the gel-stabilized liposome compositions having a uniform vesicle diameter may be obtained with active agents (the vesicle diameter is not limited to this range, the vesicle range is only controlled by the purpose for which the disclosure is to be used and limited by the kind of equipment available for its preparation). The vesicles have been shown to contain both a single lipid bilayer as well as multiple lipid bilayers.

### Example 2

### Preparation of Gel-Stabilized Liposome Vesicle System with a High Degree of Entrapment Efficiency and Stability Containing Recombinant Human Interferon α 2b (rhIFNα2b)

### (a) Materials and Methods

Method 1: A gelatin solution having a concentration of 15% w/v was prepared by dissolving 3 g of gelatin A 250 (from Sigma) in 20 ml of sterile water at 40° C. while stirring. The resultant gelatin solution was sterilized by autoclaving at 115° C. for 30 min. A lipid solution was prepared by dissolving 4 g of soybean lecithin and 0.8 g of cholesterol and 40 mg α-tocopherol in 100 ml of ether.

3.8 ml of recombinant human interferon α 2b having an activity of 6.0×10⁸ IU (Suzhou Xinbao Pharmaceutical Group, China) was added into a 3 ml aliquot of the sterilized gelatin sol, and further diluted to 15 ml with sterilized water.

The diluted gelatin solution containing rhIFNα2b was incorporated into the lipid solution and sonicated to form a homogeneous “hydrosol-in-oil” emulsion, which did not separate in 15 min after sonication. The emulsion was immediately placed into an ice-water bath at 4° C. to 10° C. to transform the inner gelatin sol into a gelatin gel core. Subsequently, the organic solvent was removed from the cooled emulsion by rotary evaporation, under reduced pressure at 4° C. to 6° C. (below the sol-gel transition temperature of the gelatin), and then 70 ml sterilized water and 15 ml of 15% (w/v) gelatin solution were added while stirring. Upon continuing to remove ether and evaporating until the last trace of ether disappear, a translucent dispersion was obtained. This dispersion was homogenized by vortexing and then sterilized by passing through a filter membrane having 0.22 μm pores. The resulting rhIFNα2b gel-stabilized liposome composition was then stored at 4 to 8° C.

Method 2: The protocol was the same as that in method 1, with the exception that the inner hydrogel core was formed from agarose (gelling temperature 37±1° C., from Shanghai, China) rather than gelatin. Agarose has a higher remelting temperature (80° C.) than gelatin, so that the gel state of the inner hydrogel core comprising agarose may be maintained at 37° C., human body temperature.

(b) Characterization of the rhIFNα2b Gel-Stabilized Liposome Vesicle System

### Vesicle Size, Trapping Efficiency and Long-Storage Stability

rhIFNα2b gel-stabilized liposome compositions prepared by the two methods previously described were characterized using various analytical techniques. A laser diffraction particle analyzer (L230, Beckman Coulter, USA) was used to determine the size of the vesicles as per the methods described in Example 1.

The effects of various loading methods on trapping efficiency of rhIFNα2b in the liposome composition were studied. To separate the “free” untrapped rhIFNα2b from the rhIFNα2b entrapped in the gel-stabilized liposomes, an aliquot of the rhIFNα2b gel-stabilized liposomes was subjected to ultracentrifugation for 2 h at 126,000×g and at a temperature of 10° C. using an ultracentrifuge. The clear supernatant was collected and diluted with 0.3% w/v Triton X-100 buffer solution (PBS at pH 7.2). An ELISA was used to determine the concentration of free rhIFN-α-2b in the supernatant.

The total amount of rhIFNα2b was determined by diluting the solution containing rhIFNα2b gel-stabilized liposomes with 0.3% Triton X-100 buffer solution (PBS, pH 7.2), incubating at 10° C. for 30 min to rupture the liposomes and release the rhIFNα2b, and then assaying for rhIFNα2b by ELISA. The trapping efficiency was calculated according to Equation 1:

$$Trapping efficiency ( \% ) = \frac{100 \times ( total amount of drug - amount of free drug )}{total amount of drug} \qquad ( 1 )$$

### Anti-Viral Activity

The antiviral activity of gel-stabilized liposomes containing rhIFN-α-2b was measured by bioassay. Briefly, rhIFN-α-2b was titrated to determine the 50% cytopathic effect reduction, using vesicular stomatitis virus and human amnionic cells (WISH). This effect was determined by measuring the cellular uptake of neutral red dyes (using an auto-reader at 570 nm). Assays employed international reference preparations for human interferon-α (obtained from the National Institute for Biological Standards and Control, Beijing, P. R. China). All titres are reported in IU·mL⁻¹.

The gel-stabilized liposome vesicle system containing rhIFN-α-2b prepared by method 1 were stored at 4˜8° C. with a pack of vial (1 ml per a vial). Samples were analyzed at indicated storage times.

### (c) Results

Table 2 shows the average size, size distribution and the encapsulation efficiency for rhIFNα2b gel-stabilized liposome composition prepared using the two methods used. Table 3 shows the stability of the gel-stabilized liposome composition containing rhIFNα2b, expressed in criteria such as vesicle sizes, entrapment efficiency, and antiviral activity, when stored at 4° C. over a period of 12 months. The results presented in Table 2 clearly show that the composition of the present disclosure represents a novel gel-stabilized liposome drug delivery system, and the preparation method involved is capable of achieving highly efficient encapsulation (up to 98%) and a narrow vesicle size distribution. The data in Table 3 show that under the storage temperature of about 4° C. to 8° C., there appear to be no significant changes in the encapsulation efficiency, vesicle size and antiviral activity of rhIFNα2b over the 12-month study period, which indicates that the gel-stabilized liposome compositions of the present disclosure possess excellent stability.

### Example 3

### Preparation of Gel-Stabilized Liposome Composition Containing Amphotericin 8 (AMB)

### (a) Materials and Methods

A gelatin solution was prepared according to the protocol of Method 1 in Example 2. A lipid solution was prepared by dissolving 3.5 g of soybean lecithin, 0.55 g of cholesterol and 40 mg of α-tocopherol, in 90 ml of ether.

0.42 g of AMB (North China Pharmaceutical Groups Corporation, China) were added to a 5.3-ml aliquot of the gelatin solution, which was diluted to 30 ml with sterile and injectable water, and the pH adjusted to a range of 5-6 with sodium succinate, and by sonication. The gelatin solution comprising AMB was incorporated into the lipid solution and sonicated at 800 W to form a homogeneous emulsion, which was then placed in an ice-water bath at 4 to 10° C. to transform the inner gelatin sol into the gelatin gel state. The organic solvent was removed from the cooled emulsion by rotary evaporation under vacuum at 4 to 8° C., and 70 ml sterilized water and additional gelatin were added while stirring. Removal of the organic solvent was continued until the last trace of it disappeared. The pH of the resultant dispersion was adjusted to the range of 5 to 6 with sodium succinate and 3 g of mannitol was added to adjust the osmotic pressure to the range of 270 to 330 mOsm. Subsequently, the resulting dispersion was homogenized using a high-pressure homogenizer system (Nanomaizer, YSNM-1500, Yoshida Kikai Co., Ltd., Japan) until a translucent dispersion was obtained. This dispersion was then sterilized using a filter membrane having pore sizes of 0.22 μm, and stored at 4 to 8° C.

### (b) Transmission Electron Micrographs (TEM), Vesicle Size and Entrapment Efficiency

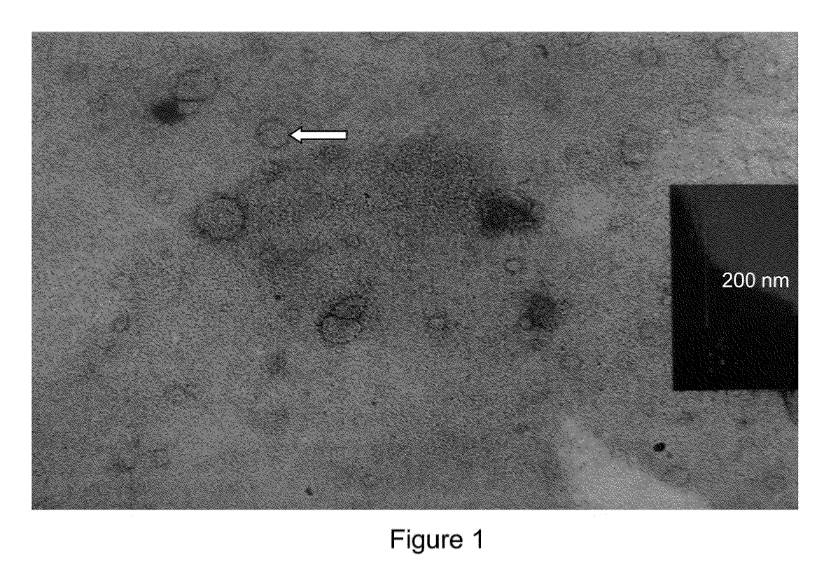

Transmission Electron Micrographs (TEM) of the gel-stabilized liposomes of the disclosure containing AMB show vesicles that have a substantially spherical morphology and a single lamillar (see FIG. 1). The vesicles also do not appear to aggregate and are separated by the external thermo reversible gel network.

The vesicle size was measured, and was found on average to be about 92±16 nm. A Sephadex G-50 gel column was used to separate free AMB from AMB entrapped in the liposome vesicles and an HPLC (Jaso1580, Japan) was used to measure encapsulated drug amount and total drug amount (Idem T. and Arican-Cellat N. Journal of chromatographic science, 2000. 38(8):338-343). The trapping efficiency was calculated according to Equation 1 and was found to be 99.3%. Experiments were performed over a storage term of 6 months at 4° C. to 8° C. to determine whether any change occurred in vesicle size, entrapment efficiency, AMB content, and pH values. No apparent changes were detected over the experimental period, which indicated that the gel-stabilized liposomes were capable of highly efficient encapsulation of AMB and excellent stability.

### (c) Pharmacokinetics and Tissue Distribution of the Gel-Stabilized Liposome Vesicle System Containing AMB

The gel-stabilized liposomes containing AMB in a concentration of about 4.2 mg/ml were prepared according to the method described above. DAMB, a commercially available amphotericin B solubilized in desoxycholate and provided as a lyophilized yellow powder, was used as a control, and was dissolved with sterile water and then further diluted with 5% glucose solution to a final concentration of 1 mg/ml. Male Wistar rats weighing from 180 to 220 g were used as animal models for studying the distribution and pharmacokinetics of gel-stabilized liposome vesicle system containing AMB compared with DAMB.

Rats were randomized into two groups (five per group) to provide pharmacokinetic evaluation. One group received a single intravenous injection of 1 mg of DAMB per kg over 1 min via a tail vein. Another group received a single intravenous dose of gel-stabilized liposomes containing AMB, providing of 1 mg of AMB per kg over 1 min via a tail vein. After dosing, blood samples were collected from five rats per group at 0.5, 1, 3, 5, 8, 12, 24 h. The plasma was separated by centrifugation, and approximately 0.5 ml was frozen at −18° C. until amphotericin B concentrations were assayed

To evaluate tissue distribution, rats were randomized into six groups (five per group). Control animals (Group 1 to Group 3) received a single intravenous dose of DAMB (1 mg/kg). Groups 4 to Group 6 received a single intravenous dose of gel-stabilized liposomes containing AMB (1 mg/kg). At 0.5 h, 4 h and 24 h following dosing, rats (five at each indicated time) were sacrificed, and liver, spleen, kidney, heart and lung, were collected. The tissue samples were blotted dry and stored frozen (−80° C.) until assayed for amphotericin B concentrations.

### (d) Assay

Amphoteracin B in blood and tissues was determined using HPLC as reported previously (Garry, 1998, Antimicrobial agents and chemotherapy: 42:263-268).

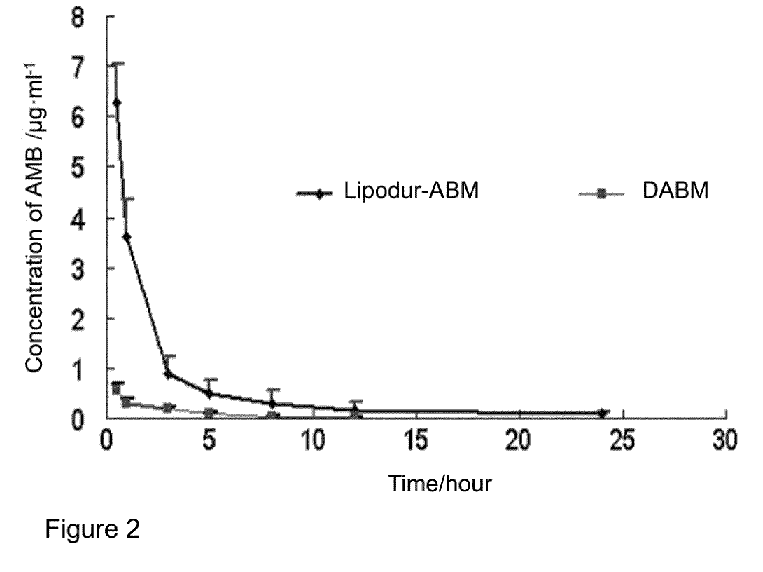

Results: The plasma concentration-versus-time curves for DAMB and gel-stabilized liposomes containing AMB, are shown in FIG. 2. The results indicate that the plasma concentration of AMB and AUC_{0-∞} after administration of gel-stabilized liposomes containing AMB are distinctly higher than those of the control, DAMB. These observations are consistent with of those of liposomal AMB previously reported (G. W. Boswell, et al. Toxicilogical profile and pharmacokinetics of unilamellar liposomal vesicle formulation of amphotericin B in rats. Antimicrob. Agents Chemother., 1998, 42(2):263-268).

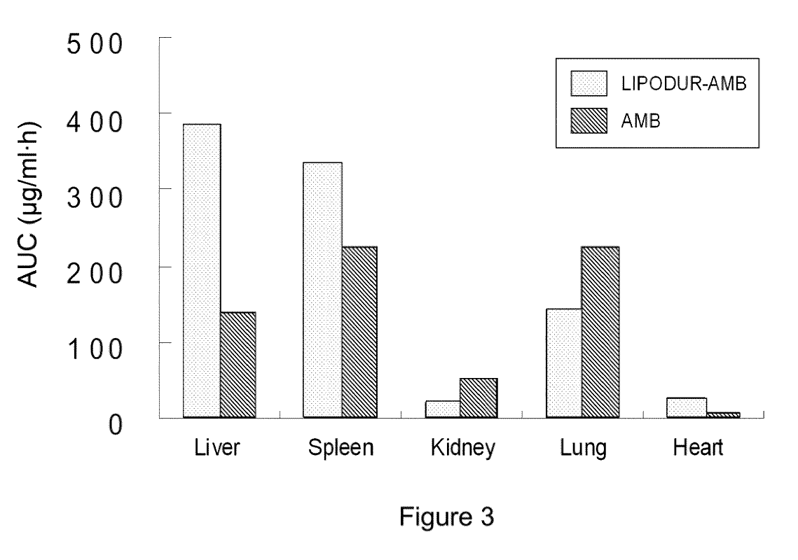

FIG. 3 shows the distribution of AMB in various tested tissues. The results obtained from this exemplary embodiment showed that the concentration of AMB obtained from administration of gel-stabilized liposomes containing AMB was significantly higher than those obtained from administration of DAMB, a control AMB formulation, in both liver and spleen, while it is significantly lower than the latter in lung, kidney and heart. The results indicated that higher amphotericin B concentrations were present in the reticuloendothelial system (RES) (spleen and liver), with lesser amounts in the non-RES (kidney and heart), which supports that the RES is a major targeting-organ for intravenous administration of gel-stabilized liposomes containing AMB.

### (e) Antifungal Activities of Gel-Stabilized Liposome Vesicle System Containing AMB

Strains: Candida albicans A₂a and Cryptococcus neoformans D₂a organisms were used to test the antifungal activity. They were provided by the Institute of Dermatology, China Academy of Medical Science.

Antifungal agents: DAMB and the gel-stabilized liposomes containing AMB were used as antifungal agents.

Antifungal susceptibility tests: The in vitro antifungal activities of DAMB and the gel-stabilized liposomes containing AMB against Candida albicans and Cryptococcus neoformans species were evaluated using standard methods.

Tests were performed using the broth dilution method. Cultures were grown on Sabouraud dextrose agar at 37° C. for 24 h until sporulation. Before inoculation for susceptibility tests, the spores were resuspended to achieve 2×10⁷ CFU/ml. A 1-ml aliquot of Sabouraud dextrose broth containing DAMB, or gel-stabilized liposomes containing AMB, was inoculated with a 100-μl aliquot of the germinated spore suspension. The cultures were then incubated for 24 h and 48 h at 37° C. The MIC was determined as the lowest concentration of, antifungal agents that inhibited visible fungal growth after the 24 h of incubation. The MFC was determined as the lowest concentration of antifungal agents that killed fungal cells after the 48 h of incubation.

The results for MIC and MFC of DAMB and the gel-stabilized liposomes containing AMB shown in Table 4 were obtained from averaging duplicate counts for each incubation period.

Gel-stabilized liposomes comprising AMB as an active agent were found to have inhibitory activity against the tested pathogenic strains of fungi. These results indicate that MIC and MFC of gel-stabilized liposomes comprising AMB are largely similar to that of DAMB, which suggests that loading amphotericin B into the gel-stabilized liposomes has no inhibitory effect on the antifungal activity of AMB in vitro.

### Example 4

### Preparation of Gel-Stabilized Liposome Vesicle System Containing Bovine Hemoglobin

### (a) Materials and Methods

Method 1: A gelatin solution having a concentration of 30% w/v was prepared by dissolving gelatin 250 A at 40° C. in sterile Tris buffer solution (pH 7.4), and sterilizing the solution by autoclaving at 115° C. for 30 min. A gelatin solution containing bovine hemoglobin was prepared by incorporating 30 ml of bovine hemoglobin in a 4-ml aliquot of the 30% gelatin solution and glycerin (in an amount to make the liposome iso-osmotic). A lipid solution was prepared by dissolving 5 g of soybean lecithin and 1.5 g of cholesterol in 180 ml of ether.

180 ml of the lipid solution was added to the gelatin solution comprising bovine hemoglobin and sonicated at 200 W to form a homogeneous emulsion, which was immediately placed into an ice-water bath to transform the inner gelatin sol into the gelatin gel state. A portion of the organic solvent, ether, was removed from the cooled emulsion through rotary evaporation under reduced pressure at 4 to 10° C.

Cooled 60 mL of 0.45% (w/v) gelatin solution, prepared by diluting the above sterilized 30% (w/v) gelatin solution with sterilized and injectable water (60 ml), was added with stirring at 4 to 10° C. Removal of the organic solvent was continued at 4 to 10° C. until the last trace of it disappeared. Sodium chloride solution was added to adjust iso-osmia. The 30% (w/v) gelatin solution was added to adjust to 3% concentration of gelatin in the external phase. Tris-HAC buffer solution was added to regulate pH to 7.4. The resulting dispersion was then passed through a filter membrane with 0.22 μm pores for sterilization. The finished product may be stored at 4 to 8° C., or spray dried or lyophilized and then stored at 4 to 8° C.

Method 2: The preparation of the gelatin solution and the gelatin solution comprising hemoglobin was identical to that of Method 1. A lipid solution was prepared by dissolving 4.5 g of soybean lecithin and 1.25 g of cholesterol in 150 ml of methyl tertiary butyl ether (MTBE). The lipid solution was incorporated into the gelatin solution comprising hemoglobin and sonicated at 200 W to form a homogeneous emulsion, which was immediately cooled in an ice-water bath to transform the internal gelatin droplets containing hemoglobin from sol into gel. A portion of the organic solvent in the emulsion was removed through rotary evaporation under vacuum at 10 to 14° C.

Cooled 60 mL of 0.45% (w/v) gelatin solution, prepared by diluting the above sterilized 30% (w/v) gelatin solution with sterilized and injectable water (60 ml), was added with stirring at 4 to 10° C. and then the last traces of organic solvent were removed under vacuum as was described earlier. 30% (w/v) gelatin solution was added to adjust the external phase to 3% gelatin. Sodium chloride solution was added to adjust iso-osmia. Tris-HAC buffer solution was added to regulate pH to 7.4 and the resulting mixture was then dispersed by vortexing or sonicating at 100 W until a semi-transparent dispersion was obtained. The dispersion was sterilized using a filter as was discussed in earlier examples. It may be stored at 4 to 8° C., or spray dried or lyophilized and then stored at 4 to 8° C.

### (b) Vesicle Size and Entrapment Efficiency

The gel-stabilized liposomes containing bovine hemoglobin prepared by the two methods described above were characterized. A laser diffraction particle analyzer (L230, Beckman Coulter, USA) was used to determine the size of the vesicles according to methods described in Example 1.

The effects of various loading methods on entrapment efficiency of bovine hemoglobin in the gel-stabilized liposomes were studied. To separate the “free” untrapped bovine hemoglobin from the bovine hemoglobin entrapped in the gel-stabilized liposomes, an aliquot of the gel-stabilized liposomes containing hemoglobin after dilution was subjected to ultracentrifugation for 2 h at 126,000×g and at a temperature of 4° C. using an ultracentrifuge. All supernatant was collected and diluted with AHD reagent containing 4% w/v Triton X-100. An alkaline haematind-575 method was used to determine the concentration of free hemoglobin in the supernatant and the total amount of hemoglobin (both free and encapsulated hemoglobin) of the original solution containing the gel-stabilized liposomes (Wolf H U, Lang W, Zander R. Clin Chim Acta, 1984, 136: 95˜104.). The trapping efficiency was calculated according to Equation 1 described in Example 1.

### (c) Results

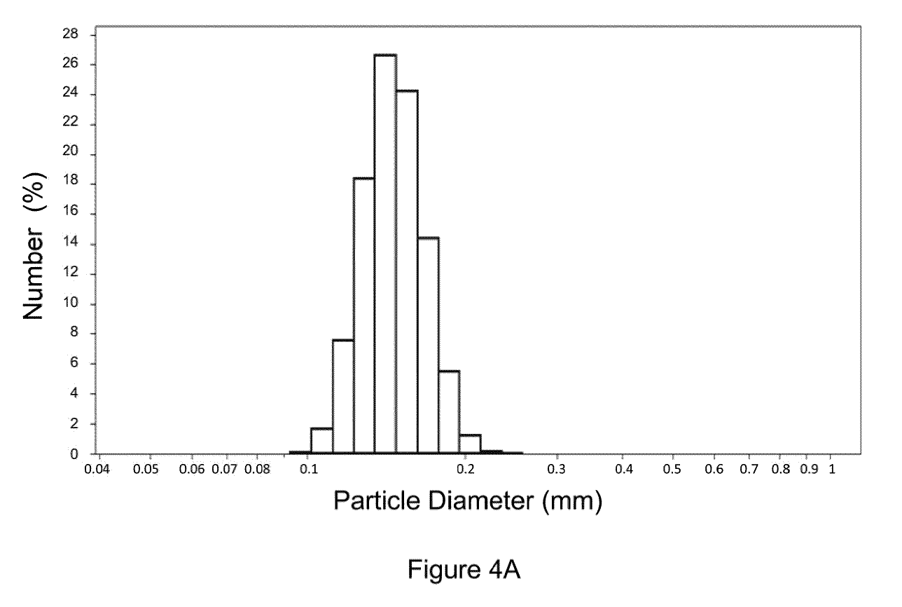

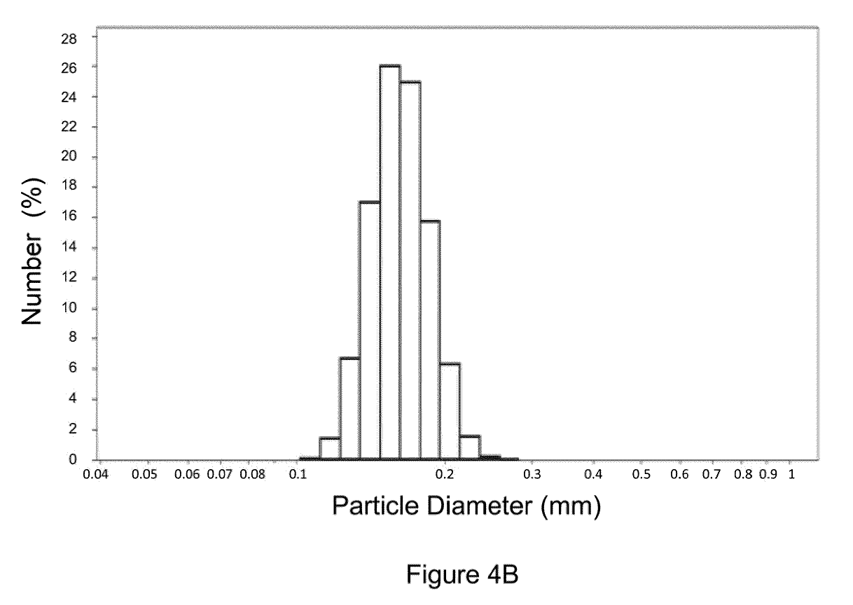

The results are shown in Table 2. The size of vesicle prepared by method 1 and by method 2 was found to be on average 147±20 nm and 163±22 nm, respectively (shown in FIGS. 4A and 4B). The amount of hemoglobin entrapped in the liposomes of the disclosure prepared using the two methods were all on average 100%.

### Example 5

### Preparation of Gel-Stabilized Liposome Vesicle System Containing Berberine Hydrochloride

### (a) Materials and Methods

A gelatin solution having a concentration of 15% w/v was prepared by dissolving 3 g of gelatin B 250 in 20 ml of sterile water at 40° C. The gelatin solution was sterilized by autoclaving at 115° C. for 30 min. A lipid solution was prepared by dissolving 4 g of soybean lecithin and 0.8 g of cholesterol in 80 ml of ether.

Berberine hydrochloride (60 mg) was added to a 4-ml aliquot of the sterilized gelatin solution and diluted with sterilized water to 15 ml to form a gelatin sol comprising berberine hydrochloride.

The gelatin solution comprising berberine hydrochloride was then incorporated into the 80 ml of the lipid solution and sonicated to form a homogeneous emulsion, which did not separate in 15 min after sonication. The emulsion was immediately placed into an ice-water bath at 4 to 10° C. to transform the gelatin solution droplets comprising berberine hydrochloride from sol into gel. The organic solvent in the cooled emulsion was removed by rotary evaporation under reduced pressure at 4 to 10° C.

Sterilized water (40 ml) and additional gelatin (2.1 g) were added while stirring. Subsequently, removal of the organic solvent was continued and under vacuum until the last trace of the organic solvent disappeared. The resulting dispersion was homogenized to form vesicles with desired size. The dispersion was sterilized by passing it through a filer membrane with pore sizes of 0.22 μm.

### (b) Characterization and Results

The vesicle size of the resulting gel-stabilized liposomes containing berberine hydrochloride was measured by laser diffraction particle analyzer as described earlier, and was found to be on average 114±23 nm.

To assess the amount of free berberine hydrochloride (i.e., not encapsulated in the liposomes), an aliquot of the gel-stabilized liposomes containing berberine hydrochloride was passed through column loaded cation exchange resin and eluted with distilled water. The collected eluant was measured at 345 nm using a UV spectrophotometer to obtain the concentration of the free drug. The total drug concentration entrapped in the gel-stabilized liposomes containing berberine hydrochloride was assessed by dissolving the vesicles comprising berberine hydrochloride in a solvent comprising Triton-X 100, alcohol and water in a volumetric ratio of 1:30:69, to release the entrapped drug. The concentration of released berberine hydrochloride was measured at 345 nm using UV spectrophotometry.

The entrapment efficiency was calculated using Equation 1. The amounts of berberine hydrochloride entrapped in the liposomes of the present disclosure was on average 96%.

### Example 6

### Preparation of Gel-Stabilized Liposome Vesicle System Containing Doxurubicin Hydrochloride

### Method 1:

### (a) Materials and Methods

A gelatin solution was prepared according to the protocol described earlier (Example 2, Method 1). A lipid solution was prepared by dissolving 4 g of soybean lecithin, 0.6 g cholesterol, 0.1 g α-tocopherol in 120 ml of ether. 200 mg of doxorubicin hydrochloride was dissolved in a 6-ml aliquot of the gelatin solution and diluted with 24 ml with sterilized water. The gelatin solution comprising doxorubicin hydrochloride was then incorporated into the 120 ml of the lipid solution, and sonicated to form a homogeneous emulsion, which was placed into an ice-water bath at 4 to 10° C. to transform the sol droplets into a gel core. A portion of the organic solvent in the emulsion was removed by rotary evaporation under vacuum at 4 to 10° C.

A 0.45% gelatin solution (70 mL), prepared by diluting the 30% gelatin solution with sterilized water, was cooled to 4-6° C. and added to the above emulsion. The organic solvents were continued to be removed under the same condition as described in earlier examples until the last trace of the organic solvents disappeared, and then the 30% gelatin solutions was added to provide an external phase with a gelatin concentration of 3%. As was discussed in the earlier examples, the dispersion may be further sterilized, and dried by lyophilization and stored at 4 to 8° C.

### (b) Characterization and Results

The vesicle size of gel-stabilized liposomes containing doxurubicin hydrochloride was measured using the techniques discussed earlier, and was found to be on average 120±24 nm.

To measure the encapsulation efficiency of the drug, an aliquot of gel-stabilized liposomes containing doxurubicin hydrochloride was passed through column loaded cation exchange resin and eluted with distilled water. The collected eluant was measured at 480 nm using a UV spectrophotometer to obtain the concentration of the free drug. The total amount of doxorubicin hydrochloride present in gel-stabilized liposomes containing doxurubicin hydrochloride was measured by dissolving the vesicles in a solvent comprising Triton-X 100, alcohol, and water in a volumetric ratio of 1:30:69, to release the entrapped drug and using UV spectrophotometric analysis at 480 nm.

The trapping efficiency was calculated according to the formula in Equation 1 and was found to be about 95.6%.

### Method 2:

### (a) Materials and Methods

The protocol was the same as that in Method 1, with the exception that the internal hydrogel core was formed from agarose gel rather than gelatin gel and organic solvent used was cyclohexane.

Lipid solution was prepared by dissolving 4 g of HSPC (SPC-3, Lipoid, Germany) 0.8 g of cholesterol in 120 ml of cyclohexane. Agarose solution having a concentration of 4% w/v was prepared by dissolving 2 g of agarose in 50 ml of sterile water at 80° C. The agarose solution was sterilized by autoclaving at 115° C. for 30 min.

Doxorubicin hydrochloride (200 mg) was dissolved in a 15 ml aliquot of the agarose solution diluted with 15 ml of sterilized water at 50° C. The agarose solution comprising doxorubicin hydrochloride was then incorporated into the 120 ml of the lipid solution, and sonicated at 45° C. to form a homogeneous emulsion, which was placed into an ice-water bath at 4 to 10° C. to transform the agarose sol droplet into a gel core. The organic solvent in the cooled emulsion was removed by rotary evaporation under reduced pressure at 35 to 40° C.

A 0.45% gelation solution (70 mL), prepared by diluting the 30% gelatin solution with sterilized water, was cooled to about 4-6° C. and added to the emulsion with stirring. The organic solvents were continued to be removed under the same conditions as described in earlier examples until the last trace of the organic solvents disappeared, and then the 30% gelatin solution was added to provide an external phase gelatin concentration of 3%, resulting in a translucent dispersion. As discussed in the earlier examples, the dispersion may be further sterilized and stored at 4 to 10° C.

### (b) Characterization and Results

The vesicle size and entrapping efficiency in the gel-stabilized liposome vesicle system containing doxurubin hydrochloride was measured using the same method discussed in method 1. The vesicle size was found to be on average 133±19 nm. The amount of doxurubin hydrochloride entrapped in the lipid vesicles was on average 98.2%.

### Method 3:

### (a) Materials and Methods

The protocol was the same as that in Method 1, with the exception that the final liposomal compositions were further homogenized with the probe sonicator at 600 W for 3 min.

### (b) Characterization and Results

The vesicle size and entrapping efficiency in gel-stabilized liposome vesicle system containing doxurubin hydrochloride was measured using the same method discussed in method 1. The vesicle size was found to be on average 133±19 nm. The amount of doxurubin hydrochloride entrapped in the lipid vesicles was on average 98.2%.

### Example 7

### Preparation of Gel-Stabilized Liposomes Containing Paclitaxol

### (a) Materials and Methods

Method 1: A gelatin solution was prepared according to the protocol described earlier (Example 2, Method 1). A lipid solution was prepared by dissolving 6 g of soybean lecithin, 0.6 g cholesterol, 50 mg α-tocopherol and 240 mg paclitaxol in 180 ml of ether. A 6-ml aliquot of the resulting gelatin solution was diluted with 24 ml with sterilized water. 25 ml of the diluted gelatin solution was incorporated into the 180 ml of the lipid solution, and sonicated to form a homogeneous emulsion, which was placed into an ice-water bath at 4 to 10° C. to transform the sol droplet into a gel core. The organic solvent in the emulsion was removed by rotary evaporation under vacuum at 4 to 10° C.

Sterilized water (75 ml) and the gelatin (2.1 g) were added while stirring. Removal of the organic solvent was continued under the same condition as described in earlier examples until the last trace of the organic solvent disappeared, Subsequently, the resulting dispersion was homogenized using a high-pressure homogenizer system (Nanomaizer, YSNM-1500, Yoshida Kikai Co., Ltd., Japan) until a translucent dispersion was obtained. As discussed in the earlier examples, the dispersion may be further sterilized, and stored at 4 to 10° C.

### (b) Characterization and Results

Vesicle size of the gel-stabilized liposomes containing paclitaxol was measured using the techniques discussed earlier, and was found to be on average 131±20 nm.

To measure encapsulation efficiency of drug, free and entrapped paclitaxol, the liposomes were separated by Sephadex G50 column. Free and the total amount of drug present in gel-stabilized liposomes containing paclitaxol after disrupting the liposome to release encapsulated drug with methanol were analyzed by HPLC. The trapping efficiency was calculated according to Equation 1 and was found to be about 99.2%.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent disclosures are herein incorporated by reference in their entirety to the same extent as if each individual publication, patent or patent disclosure was specifically and individually indicated to be incorporated by reference in its entirety. Where a term in the present disclosure is found to be defined differently in a document incorporated herein by reference, the definition provided herein is to serve as the definition for the term.

TABLE 1

| | Gel-Forming | | | Vesicle |
|---|---|---|---|---|
| | Agent | | | Diameter |
| Expt. | (interior/ | Active | Encapsulation | (Mean ± SD)) |
| No. | exterior*) | Agent | (%) | (nm) |
| 1 | Gelatin/gelatin | — | — | 101 ± 33 |
| 2(1) | Gelatin/gelatin | RhFNα 2b | 96.2 | 96 ± 17 |
| 2(2) | Agarose/gelatin | RhFNα 2b | 97.4 | 98 ± 18 |
| 3 | Gelatin/gelatin | AMB | ^{∓}99.3 | 92 ± 16 |
| 4(1) | Gelatin/gelatin | Hemoglobin | 100 | 147 ± 20 |
| 4(2) | Gelatin/gelatin | Hemoglobin | 100 | 163 ± 22 |
| 5 | Gelatin/gelatin | Berberine | 96.0 | 114 ± 23 |
| | | hydrochloride | | |
| 6(1) | Gelatin/gelatin | Doxorubicin | 95.6 | 120 ± 24 |
| 6(2) | Agarose/gelatin | hydrochloride | 98.2 | 133 ± 19 |
| 6(3) | Gelatin/gelatin | | 91.7 | 94 ± 17 |
| 7 | Gelatin/gelatin | Paclitaxol | 99 | 131 ± 20 |

*Solvated in water (i.e., gelatin sol) and concentration of gelatin in exterior hydrogel is 3%
^{∓}Other additives were used (see detailed protocol)

TABLE 2

| | Vesicle Size | Entrapment Efficiency |
|---|---|---|
| Preparation Method | (mean ± SD)(nm) | for rhIFNα2b (%) |
| 1 | 96 ± 17 | 96.2 |
| 2 | 98 ± 18 | 97.4 |

TABLE 3

| | Average Vesicle | Encapsulation | |
|---|---|---|---|
| Time | Diameter | Efficiency | Activity |
| (month) | (nm) | (%) | (×10⁶ IU/ml) |
| 0 | 101 ± 17 | 98.8 | 6.6 |
| 1 | 99 ± 32 | 97.7 | 6.5 |
| 2 | Not determined | 98.2 | 6.6 |
| 3 | 101 ± 34 | 98.7 | 6.8 |
| 6 | 101 ± 33 | 98.4 | 6.2 |
| 12 | 103 ± 35 | 97.9 | 6.3 |

TABLE 4

| | Antifungal | | vesicle of the |
|---|---|---|---|
| Strain | activity | DAMB | disclosure |
| Candida albicans | MIC/mg · L⁻¹ | 1.00 | 0.63 |
| | MFC/mg · L⁻¹ | 1.00 | 0.25 |
| Cryptococcus | MIC//mg · L⁻¹ | 1.00 | 2.00 |
| neoformans | MFC//mg · L⁻¹ | 1.00 | 2.00 |

1. A gel-stabilized liposome composition comprising: liposomes having an internal phase; and an external phase, wherein the internal phase comprises an internal thereto-transformable hydrogel and the external phase comprises an external thermo-reversible hydrogel and the liposomes are dispersed in the external phase.

2. The gel-stabilized liposome composition according to claim 1, wherein the internal thermo-transformable hydrogel and the external thermo-reversible hydrogel are natural, semi-synthetic or synthetic hydrogels and/or are biodegradable and/or biocompatible.

3. The gel-stabilized liposome composition according to claim 1 wherein the hydrogels for the internal thermo-transformable hydrogel or external thermo-reversible hydrogel are selected from gelatin and agarose and mixtures thereof.

4. The gel-stabilized liposome composition according to claim 3, wherein the hydrogels for the internal thermo-transformable hydrogel or external thermo-reversible hydrogel are both gelatin.

5. The gel-stabilized liposome composition according to claim 3, wherein the hydrogel for the internal thermo-transformable hydrogel is agarose.

6. The gel-stabilized liposome composition according to claim 1, wherein one or more agents are encapsulated into the liposomes.

7. The gel-stabilized liposome composition according to claim 1, wherein the liposomes are characterized by lipid bilayers or multilayers.

8. The gel-stabilized liposome composition according to claim 7, wherein water-soluble agents are encapsulated within the internal thermo-transformable hydrogel and lipid-soluble agents are encapsulated within the lipid bilayer or multilayers of the liposomes.

9. The gel-stabilized liposome composition according to claim 1, wherein the liposomes are formed from one or more lipids selected from phospholipids, stearylamines, fatty acids and fatty acid amides.

10. The gel-stabilized liposome composition according to claim 9, wherein the liposomes are formed from phospholipids selected from soybean lecithin, egg lecithin, lecithin, lysolecithin, phosphatidylserine, phosphatidylethanolamine, phosphatidylcholine and phosphatidylinositol.

11. The gel-stabilized liposome composition according to claim 9, wherein the liposomes are formed from phospholipids and further wherein the phospholipids are mixed with a sterol.

12. A pharmaceutical composition comprising a gel-stabilized liposome composition according to claim 1 and a pharmaceutically acceptable carrier.

13. A process for preparing a gel-stabilized liposome composition according to claim 1 comprising:
(a) preparing or obtaining a solution comprising one or more internal thermo-transformable hydrosols and, optionally, at least one water-soluble active agent wherein the hydrosol is prepared in an aqueous medium;
(b) preparing or obtaining a solution comprising one or more lipids and, optionally, one or more lipid-soluble active agents in an organic solvent that is substantially immiscible with the aqueous medium;
(c) combining the solution of (a) with the solution of (b) at a temperature which is higher than the sol-gel phase transition temperature of the one or more internal thermo-transformable hydrosols and under conditions to produce an emulsion;
(d) lowering the temperature of said emulsion of (c) to below the sol-gel phase transition temperature of the one or more internal thermo-transformable hydrosols to transform said one or more hydrosols into one or more hydrogels in said emulsion;
(e) optionally removing a portion of the organic solvent from the emulsion of (d) at a temperature lower than the sol-gel phase transition temperature of the one or more internal thermo-transformable hydrogels; and
(f) combining the emulsion of (d) or (e) with one or more external thermo-reversible hydrogels and removing any remaining organic solvent, wherein said combining and said removal of solvent is at a temperature lower than the sol-gel phase transition temperature of the one or more internal thermo-transformable hydrogels and under conditions to form a homogeneous dispersion of liposomes in the one or more external thermo-reversible hydrogels, wherein said one or more external thermo-reversible hydrogels are prepared in an aqueous medium and the liposomes have an internal phase comprised of the one or more internal thermo-transformable hydrogels.

14. The process according to claim 13, wherein the organic solvents in (b) is selected from solvents in which the lipids are substantially soluble and which are substantially immiscible with the aqueous media.

15. The process according to claim 14, wherein the organic solvents in (b) is selected from diethyl ether, di-n-butyl ether, methyl tertiary butyl ether (MTBE), cyclohexane and chloroform and combinations thereof.

16. The process according to claim 13, wherein the conditions to produce an emulsion in (c) comprise adding the solution comprising one or more internal thermo-transformable hydrosols into the solution comprising one or more lipids in a suitable ratio, followed by a mechanical dispersion to form an emulsion.

17. The process according to claim 13, wherein the solution comprising one or more lipids is used in amounts excess to the solution comprising one or more internal thermo-transformable hydrosols.

18. The process according to claim 13, wherein the emulsion of (c) is a hydrosol-in-oil emulsion in which the hydrosol from (a) is dispersed in the organic solvent in the form of individual droplets.

19. The process according to claim 13, wherein the organic solvent is at least partially removed after formation of emulsion of (d).

20. The process according to claim 19, wherein the removal of a portion of the organic solvent is done at a temperature below the sol-gel phase transition temperature of the one or more internal thermo-reversible hydrogels.

21. The method according to claim 19, wherein sufficient organic solvent is removed to obtain a volume ratio of the emulsion of (d) to the aqueous medium comprising the one or more external thermo-reversible hydrogels in the range of about 3:7 to about 8:2.

22. The method according to claim 19, wherein addition of further external thermo-reversible hydrogel in aqueous medium is performed following evaporation of a portion of the organic solvent from the emulsion of (d).

23. The method according to claim 22, wherein the concentration of the further external thermo-reversible hydrogel in the aqueous medium is in the range of about 0% to about 1% (w/v).

24. The method according to claim 22, wherein any remaining organic solvent is removed following the addition of the further external thermo-reversible hydrogel in aqueous medium.

25. The method according to claim 22, wherein, following removal of the remaining organic solvent, a final aqueous medium comprising external thermo-reversible hydrogel is added, the final aqueous medium having an external thermo-reversible hydrogel concentration in the range of about 20% to about 40% (w/v), at a temperature below the sol-gel phase transition temperature of the one or more internal thermo-transformable hydrosols, to provide a final external hydrogel concentration in the liposome composition of about 2% to about 5% (w/v).

26. A method for delivering one or more agents to a biological system comprising administering a gel-stabilized liposome composition according to claim 6 to said system.

27. A method of delivering an active agent to a subject in need of treatment with the active agent comprising administering an effective amount of a gel-stabilized liposome composition according to claim 6 to said subject, wherein the agent is an active agent.
